(19) United States
(12) Plant Patent Application Publication
(10) Pub. No.: US 2011/0107482 P1
(43) Pub. Date: May 5, 2011

(54) Strawberry plant named 'DrisStrawFourteen'

(50) ;

(75) Inventors: Philip J. Stewart, Watsonville, CA (US); Martin P. Madesko, Watsonville, CA (US); JoAnne F. Coss, Watsonville, CA (US); Bruce D. Mowrey, Watsonville, CA (US)

(73) Assignee: Driscoll Strawberry Associates, Inc., Watsonville, CA (US)

(21) Appl. No.: 12/590,071

(22) Filed: Nov. 2, 2009

(51) Int. Cl. A01H 5/00

(52) U.S. Cl. PLT/208

(57) ABSTRACT

This invention relates to a new and distinct cultivar of strawberry plant named ‘DrisStrawFourteen’. The new cultivar is primarily characterized by a medium density plant with medium vigor, medium-sized fruit having a large hollow center, and a partially everbearing habit, is disclosed.

### GENUS AND SPECIES

Fragaria×ananassa

### VARIETY DENOMINATION

‘DrisStrawFourteen’

### BACKGROUND OF THE NEW PLANT

The present invention relates to a new and distinct strawberry cultivar designated ‘DrisStrawFourteen’ and botanically known as Fragaria×ananassa. This new strawberry cultivar was discovered in Monterey County, Calif. in May 2005 and originated from a cross between the proprietary female parent ‘159K312’ (unpatented) and the proprietary male parent ‘128K296’ (unpatented). The original seedling of the new cultivar was first asexually propagated by stolons or tissue culture at a nursery in Shasta County, Calif.

‘DrisStrawFourteen’ was subsequently asexually propagated in Shasta County, Calif. and underwent further testing in Monterey County, Calif. for five years. The present invention has been found to retain its distinctive characteristics through successive asexual propagations via stolons and tissue culture.

### DESCRIPTION OF THE PHOTOGRAPHS

The accompanying color photographs show typical specimens of the new cultivar at various stages of development as nearly true as it is possible to make in color reproductions. The photographs were taken from 9-month-old plants.

FIG. 1 shows overall plant habit including fruit at various stages of development.

FIG. 2 shows leaves of the plant with three leaflets.

FIG. 3 shows both the upper side and underside of several of the flowers.

FIG. 4 shows the whole fruit.

FIG. 5 shows the fruit in longitudinal cross-section.

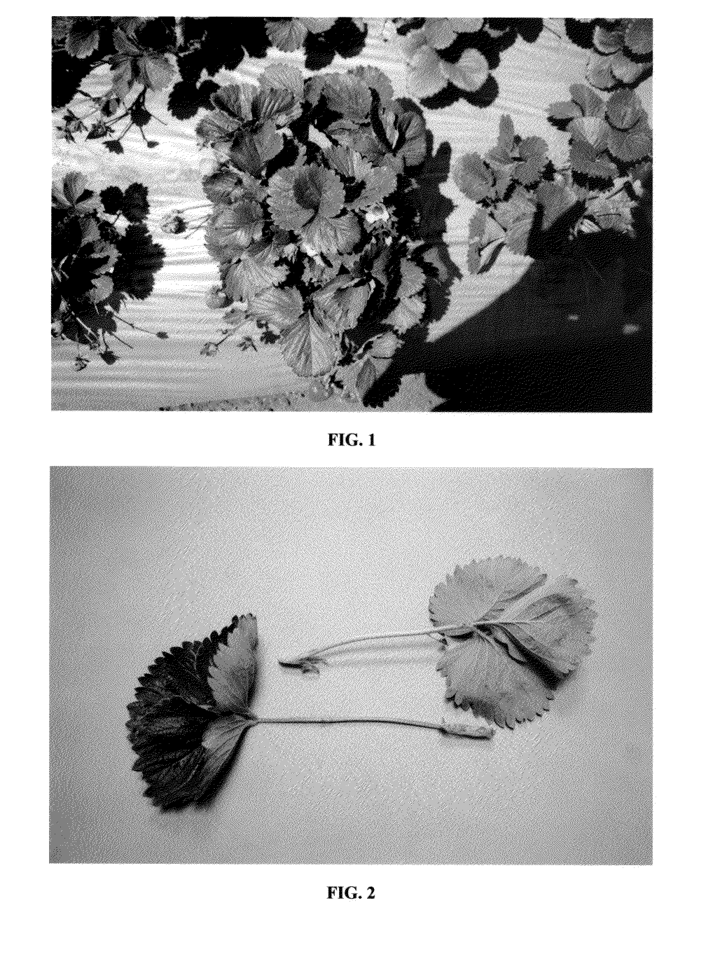

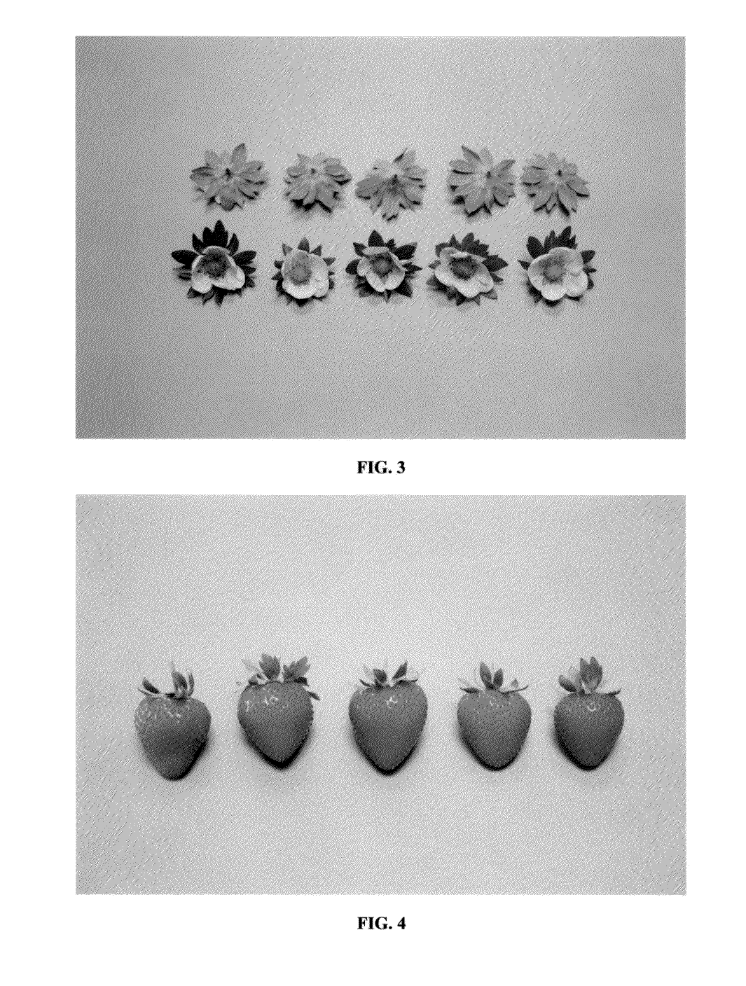

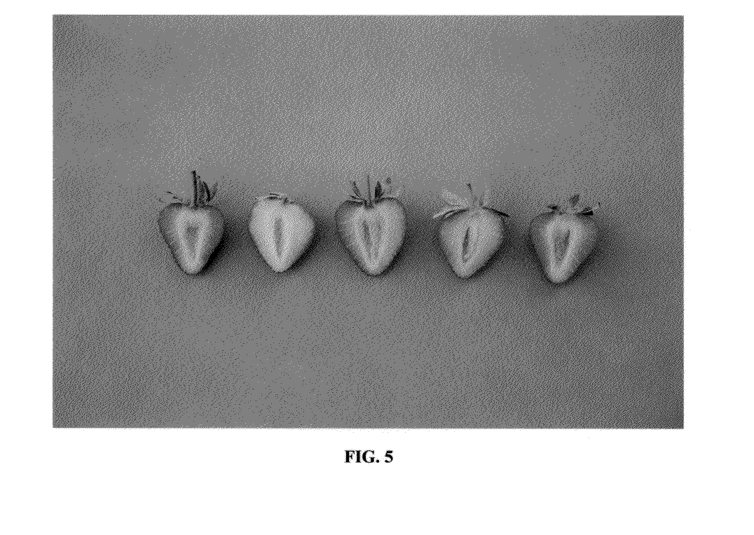

### DESCRIPTION OF THE NEW CULTIVAR

The following description of ‘DrisStrawFourteen’ is based on observations taken in Monterey County, Calif. from 2005 to 2009. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFourteen’ has not been observed under all possible environmental conditions. The botanical description of ‘DrisStrawFourteen’ was taken from 9-month-old plants and the botanical descriptions of the comparison varieties, ‘San Juan’ (U.S. Plant Pat. No. 12,899) and ‘Driscoll Lanai’ (U.S. Plant Pat. No. 15,145), were taken from 10-month-old plants and from 8-month-old plants, respectively. Color terminology follows The Royal Horticultural Society Colour Chart, London (R.H.S.) (2001).

### DETAILED BOTANICAL DESCRIPTION

Table 1 shows plant characteristics of the new variety compared with plant characteristics of the commercial varieties ‘San Juan’ and ‘Driscoll Lanai’. Plant characteristics include plant height, diameter, number of crowns per plant, habit, the density of individual plants and the vigor.

TABLE 1

| Characteristic | ‘DrisStrawFourteen’ | ‘San Juan’ | ‘Driscoll Lanai’ |
|---|---|---|---|
| Plant height (cm) | 24.9 | 23.6 | 22.8 |
| Plant diameter (cm) | 39.6 | 44.2 | 40.1 |
| Number of crowns/ | 3 | 2 | 2 |
| plant | | | |
| Habit | Globose | Globose | Globose |
| Density of | Medium | Dense | Medium |
| individual plant | | | |
| Vigor | Medium | Strong | Medium |

Table 2 shows leaf characteristics of the new cultivar compared with leaf characteristics of ‘San Juan’ and ‘Driscoll Lanai’. Leaf characteristics include terminal leaflet length and width in centimeters, length to width ratio, number of teeth per terminal leaflet, shape of teeth, color of upper side and underside of leaf, leaf shape in cross section, leaf blistering, leaf glossiness, number of leaflets, terminal leaflet margin, terminal leaflet length to width ratio, overall leaf shape and shape of leaf apex and base.

TABLE 2

| | ‘DrisStrawFourteen’ | ‘San Juan’ | ‘Driscoll Lanai’ |
|---|---|---|---|
| Leaf | | | |
| Characteristic | | | |
| Terminal leaflet | 7.6 | 7.4 | 7.2 |
| length (cm) | | | |
| Terminal leaflet | 0.84 | 0.74 | 0.69 |
| width (cm) | | | |
| Terminal leaflet | 0.9 | 1.0 | 1.0 |
| length/width ratio | | | |
| No. teeth/ | 28 | 21 | 20 |
| terminal leaflet | | | |
| Characteristic | | | |
| Shape of teeth | Obtuse | Rounded | Rounded |
| Color of upper | RHS 147A | RHS 139A | RHS 147A |
| surface of leaf | (Dark yellow- | (Dark green) | (Dark yellow- |
| | green) | | green) |
| Color of lower | RHS 148B | RHS 147B | RHS 148C |
| surface of leaf | (Medium | (Medium | (Medium |
| | yellow-green) | yellow-green) | yellow-green) |
| Leaf shape in | Slightly | Slightly | Flat |
| cross section | concave | concave | |
| Leaf blistering | Medium | Medium | Medium |
| Leaf glossiness | Medium | Medium | Medium |
| No. leaflets | 3 only | 3 only | 3 only |
| Terminal leaflet | Flat | Revolute to | Revolute to |
| margin | | flat | flat |
| Terminal leaflet | As long as | As long as | As long as |
| length/width ratio | broad | broad | broad |
| Terminal leaflet | Orbicular | Orbicular | Orbicular |
| shape | | | |
| Terminal leaflet | Rounded | Rounded | Rounded |
| base shape | | | |
| Terminal leaflet | Rounded | Rounded | Rounded |
| apex shape | | | |

Table 3 shows information about the petiole, the petiolule, the bract, and the stipule of the new cultivar compared to ‘San Juan’ and ‘Driscoll Lanai’. This includes petiole length in centimeters, petiole diameter in centimeters, petiole pubescence, pose of hairs on the petiole, color of the petiole, color of the petiolule, petiolule length in centimeters, petiolule diameter in centimeters, bract frequency per petiole, stipule length in centimeters, stipule width in centimeters, stipule pubescence and stipule anthocyanin coloration.

TABLE 3

| Characteristic | ‘DrisStrawFourteen’ | ‘San Juan’ | ‘Driscoll Lanai’ |
|---|---|---|---|
| Petiole length | 13.8 | 16.3 | 14.4 |
| (cm) | | | |
| Petiole diameter | 0.333 | 0.314 | 0.280 |
| (cm) | | | |
| Petiole | Sparse | Medium | Medium |
| pubescence | | | |
| Petiole pose | Upwards to | Outwards | Between |
| of hairs | downwards | | upwards |
| | | | and outwards |
| Petiole color | RHS 144B | RHS 146B | RHS 144C |
| | (Medium | (Medium | (Medium |
| | yellow-green) | yellow-green) | yellow-green) |
| Petiolule color | RHS 144B | RHS 146C | RHS 144C |
| | (Medium | (Medium | (Medium |
| | yellow-green) | yellow-green) | yellow-green) |
| Petiolule length | 7.21 | 8.63 | 12.26 |
| (mm) | | | |
| Petiolule | 0.165 | 0.170 | 0.145 |
| diameter (cm) | | | |
| Bract frequency | 1 | 1 | 0 |
| Stipule length | 3.8 | 3.3 | 3.3 |
| (cm) | | | |
| Stipule width | 0.694 | 0.929 | 0.897 |
| (cm) | | | |
| Stipule | Dense | Medium | Dense |
| pubescence | | | |
| Stipule | RHS 145D | RHS 149D | RHS 149D |
| anthocyanin | (Light | (Light | (Light |
| coloration | yellow-green) | yellow-green) | yellow-green) |

Table 4 shows stolon characteristics of the new cultivar compared to ‘San Juan’ and ‘Driscoll Lanai’. These characteristics include the number of stolons, average number of daughter plants, the anthocyanin coloration of the stolons, the thickness of the stolons, and the pubescence of the stolons.

TABLE 4

| Characteristic | ‘DrisStrawFourteen’ | ‘San Juan’ | ‘Driscoll Lanai’ |
|---|---|---|---|
| Stolon number | Medium | Between | Many |
| | | medium | |
| | | and many | |
| Average number | 30+ | 57 | 67 |
| of daughter plants | | | |
| Stolon | RHS 60C | RHS 59A | RHS 60A |
| anthocyanin | (Dark red- | (Dark red- | (Dark red- |
| | purple) | purple) | purple) |
| Stolon thickness | Medium | Medium | Between |
| | | | medium |
| | | | and thick |
| Stolon pubescence | Medium | Medium | Dense |

Table 5 shows inflorescence characteristics of the new cultivar compared to ‘San Juan’ and ‘Driscoll Lanai’. These characteristics include inflorescence position relative to foliage, time of flowering, relative flower size, flower diameter in centimeters (measured from petal tip to petal tip), petal shape, relative spacing of petals, petal apex, base and margin, petal length in centimeters, petal width in centimeters, petal length to width ratio, number of petals, petal color, calyx diameter in centimeters (measured on back of flower from sepal tip to sepal tip), diameter of calyx relative to corolla, diameter of inner calyx relative to outer, sepal shape, apex and margin, sepal length in centimeters (measured from sepal tip to point of attachment to receptacle), sepal width in centimeters, number of sepals, receptacle color and anther color.

TABLE 5

| Characteristic | ‘DrisStrawFourteen’ | ‘San Juan’ | ‘Driscoll Lanai’ |
|---|---|---|---|
| Inflorescence | Level with | Beneath | Level with |
| position | | | |
| relative to foliage | | | |
| Time of flowering | Between | Between | Between |
| (50% of plants | early and | early and | early and |
| at first flower) | medium | medium | medium |
| Flower size | Medium | Medium | Medium |
| Flower diameter | 2.430 | 2.792 | 2.540 |
| (cm) | | | |
| Petal shape | Orbicular | Orbicular | Orbicular |
| Petal spacing | Overlapping | Overlapping | Overlapping |
| Petal apex shape | Rounded | Rounded | Rounded |
| Petal margin | Entire | Entire | Entire |
| Petal base shape | Concave- | Concave- | Rounded |
| | convex | convex | |
| Petal length (cm) | 1.216 | 1.347 | 1.221 |
| Petal width (cm) | 1.149 | 1.469 | 1.285 |
| Petal length/ | As long as | As long as | As long as |
| width ratio | broad | broad | broad |
| Petal length/ | 1.1 | 0.9 | 1.0 |
| width ratio | | | |
| Typical and | 6 | 6 | 7 |
| observed petal | | | |
| number | | | |
| Petal color | RHS 155C | RHS 155D | RHS 155B |
| | (White) | (White) | (White) |
| Calyx diameter | 3.444 | 3.631 | 3.228 |
| (cm) | | | |
| Calyx diameter | Larger | Larger | Larger |
| relative to corolla | | | |
| Inner calyx | Same size | Same size | Same size |
| diameter relative | | | |
| to outer | | | |
| Sepal shape | Oval | Oval | Oval |
| Sepal apex shape | Convex | Convex | Convex |
| Sepal margin | Entire | Entire | Entire |
| Sepal length (cm) | 1.369 | 1.461 | 1.150 |
| Sepal width (cm) | 0.681 | 0.767 | 0.461 |
| Typical and | 13 | 13 | 13 |
| observed sepal | | | |
| number | | | |
| Receptacle color | RHS 2A | RHS 151A | RHS N144B |
| | (Medium yellow) | (Medium | (Medium |
| | | yellow-green) | yellow-green) |
| Anther color | RHS 152B | RHS 178A | RHS 163A |
| | (Medium | (Dark greyed- | (Medium |
| | yellow-green) | red) | greyed-orange) |

Table 6 shows fruit characteristics of the new cultivar compared to ‘San Juan’ and ‘Driscoll Lanai’. These characteristics include fruiting truss length in centimeters, fruiting truss diameter, number of berries per truss, fruiting truss attitude, fruiting truss color, fruit length in centimeters, fruit truss width in centimeters, fruit length to width ratio, fruit hollow length and width in centimeters, fruit hollow length to width ratio, fruit weight in grams, relative fruit size, predominant fruit shape, difference in shape between primary and secondary fruits, band without achenes, unevenness of fruit surface, fruit skin color, evenness of fruit color, fruit glossiness, insertion of achenes, achene coloration (sunward and shaded sides of berry) and the number of achenes per berry.

TABLE 6

| Characteristic | ‘DrisStrawFourteen’ | ‘San Juan’ | ‘Driscoll Lanai’ |
|---|---|---|---|
| Fruiting truss | 18.8 | 22.8 | 17.2 |
| length (cm) | | | |
| Fruiting truss | Medium | Between | Medium |
| length (general) | | medium | |
| | | and long | |
| Fruiting truss | 0.429 | 0.391 | 0.327 |
| diameter (cm) at | | | |
| base of truss | | | |
| Number of berries | 4 | 4 | 4 |
| per fruiting truss | | | |
| Fruiting truss | Prostrate | Prostrate | Prostrate |
| attitude | | | |
| Fruiting truss | RHS 144A | RHS 144A | RHS 144A |
| color at base | (Medium | (Medium | (Medium |
| of truss | yellow-green) | yellow-green) | yellow-green) |
| Fruit length (cm) | 4.149 | 4.349 | 3.752 |
| Fruit width (cm) | 3.840 | 3.784 | 3.683 |
| Fruit length/ | 1.1 | 1.1 | 1.0 |
| width ratio | | | |
| Fruit hollow | 2.139 | 1.837 | 1.222 |
| length (cm) | | | |
| Fruit hollow | 0.716 | 0.538 | 0.424 |
| width (cm) | | | |
| Fruit hollow | 3.0 | 3.4 | 2.9 |
| length/width ratio | | | |
| Fruit weight (g) | 29.1 | 25.4 | 24.9 |
| Fruit ratio of | As long as | As long as | As long as |
| length/maximum | broad | broad | broad |
| width | | | |
| Relative fruit size | Medium | Medium | Medium |
| Predominant | Conical | Conical | Conical and |
| fruit shape | | | almost |
| | | | cylindrical |
| Difference in shape | Slight | Slight | Slight |
| between primary | | | |
| and secondary fruits | | | |
| Band without | Absent or very | Broad | Narrow |
| achenes | narrow | | |
| Unevenness of | Weak | Medium | Weak |
| fruit surface | | | |
| Fruit skin color | RHS 46B | RHS 46A | RHS 46A |
| | (Dark red) | (Dark red) | (Dark red) |
| Evenness of | Even | Slightly | Even |
| fruit color | | uneven | |
| Fruit glossiness | Medium | Medium | Medium |
| Insertion of | Below surface | Level with | Above surface |
| achenes | to above surface | surface | |
| Achene coloration | RHS 185A | RHS 183C | RHS 176A |
| (sunward side of | (Dark greyed- | (Dark greyed- | (Dark greyed- |
| berry) | purple) | purple) | orange) |
| Achene coloration | RHS N144B | RHS 152C | RHS 152C |
| (shaded side of | (Medium | (Medium | (Medium |
| berry) | yellow-green) | yellow-green) | yellow-green) |
| Achenes per berry | 340.6 | 273.4 | 294.5 |

Table 7 shows fruit characteristics of the new cultivar compared to ‘San Juan’ and ‘Driscoll Lanai’. These characteristics include the harvest maturity, insertion of calyx, pose of calyx segments, size of calyx in relation to fruit, adherence of calyx, firmness of flesh, color of the fruit flesh, evenness of the flesh color, distribution of flesh color, hollow center, sweetness of fruit, acidity of fruit, texture of fruit when tasted, type of bearing, grams of fruit per plant.

TABLE 7

| Characteristic | ‘DrisStrawFourteen’ | ‘San Juan’ | ‘Driscoll Lanai’ |
|---|---|---|---|
| Harvest | Late March | Late March | Late March |
| interval | to early | to early | to early |
| | November | November | November |
| Harvest | Late | Between | Between |
| maturity | | early and | early and |
| | | mid-season | mid-season |
| Insertion of | Level | Set above | Level |
| calyx | | fruit | |
| Pose of calyx | Reflexed | Reflexed | Reflexed |
| segments | | | |
| Size of calyx | From same | Same size | From same |
| in relation to | size to | | size to |
| fruit | larger | | larger |
| Adherence of | Strong | Strong | Medium |
| calyx | | | |
| Firmness of | Firm | Medium | Medium |
| flesh | | | |
| Color of | RHS 155D | RHS 155B | RHS 155C |
| the flesh | (White) and | (White) and | (White) and |
| | RHS 43B | RHS 43A | RHS 46A |
| | (Medium red) | (Medium red) | (Dark red) |
| Evenness of | Slightly | Slightly | Slightly |
| flesh color | uneven | uneven | uneven |
| Distribution | Marginal | Marginal | Marginal |
| of flesh color | and central | and central | and central |
| Hollow center | Large | Small | Small |
| Sweetness | Medium | Medium | Medium |
| Acidity | Medium | Medium | Medium |
| Texture when | Medium | Medium | Medium |
| tasted | | | |
| Type of bearing | Partially | Partially | Partially |
| | everbearing | everbearing | everbearing |
| Grams of fruit/ | 1,463 | 1,303 | 1,699 |
| plant | | | |

Table 8 shows pest, stress and disease characteristics of the new cultivar compared to ‘San Juan’ and ‘Driscoll Lanai’.

TABLE 8

| Pest, Stress, | | | |
|---|---|---|---|
| or Disease | ‘DrisStrawFourteen’ | ‘San Juan’ | ‘Driscoll Lanai’ |
| Botrytis | Moderately | Susceptible | Susceptible |
| fruit rot | resistant | | |
| Powdery | Between resistant | Susceptible | Susceptible |
| mildew | and moderately | | |
| | resistant | | |
| Verticillium | Moderately | Susceptible | Moderately |
| wilt | resistant | | susceptible |
| Rain | Between resistant | Between resistant | Moderately |
| | and moderately | and moderately | resistant |
| | resistant | resistant | |

### COMPARISON WITH PARENTAL AND COMMERCIAL CULTIVARS

When ‘DrisStrawFourteen’ is compared to the proprietary female parent ‘159K312’ (unpatented), ‘DrisStrawFourteen’ has larger fruit and better organic performance than does ‘159K312’.

When ‘DrisStrawFourteen’ is compared to the proprietary male parent ‘128K296’ (unpatented), ‘DrisStrawFourteen’ has larger fruit and better organic performance than does ‘128K296’.

When ‘DrisStrawFourteen’ is compared to the commercial variety ‘San Juan’, ‘DrisStrawFourteen’ has softer but more fruit in the second half of the season than does ‘San Juan’.

Table 9 shows selected plant characteristics of the new variety compared with plant characteristics of ‘San Juan’ (U.S. Plant Pat. No. 12,899) and ‘Driscoll Lanai’ (U.S. Plant Pat. No. 15,145). Plant characteristics include terminal leaflet margin profile, insertion of achenes, and fruit production.

TABLE 9

| Characteristic | ‘DrisStrawFourteen’ | ‘San Juan’ | ‘Driscoll Lanai’ |
|---|---|---|---|
| Terminal leaflet | Flat | Revolute | Revolute |
| margin profile | | to flat | to flat |
| Insertion of | Below surface to | Level with | Above |
| achenes | above surface | surface | surface |
| Fruit production, | 1,463 | 1,303 | 1,699 |
| grams/plant | | | |

We claim:

1. A new and distinct cultivar of strawberry plant as described and shown herein.
